(19) United States
(12) Patent Application Publication
(10) Pub. No.: US 2005/0025847 A1
(43) Pub. Date: Feb. 3, 2005

(54) Microbicidal compositions and method of use

(75) Inventors: Florence Camus-Bablon, Seattle, WA (US); Lourens J.D. Zaneveld, Sao Paulo (BR); Donald Waller, Chicago, IL (US)

Correspondence Address: STERNE, KESSLER, GOLDSTEIN & FOX PLLC, 1100 NEW YORK AVENUE, N.W., WASHINGTON, DC, 20005, US

(73) Assignee: Program for Appropriate Technology in Health, Seattle, WA (US)

(21) Appl. No.: 10/786,561

(22) Filed: Feb. 26, 2004

Related U.S. Application Data
(60) Provisional application No. 60/449,936, filed on Feb. 26, 2003.

(51) Int. Cl. A61K 35/78; A61K 47/00

(52) U.S. Cl. 424/764; 514/769

(57) ABSTRACT

The present invention is directed to microbicidal compositions and methods of use thereof for preventing the transmission of or treating sexually transmitted infections and/or common vaginal infections, while minimizing disruptions to vaginal ecology. The microbicidal compositions of the present invention comprise the microbicidal agent bisabolol. In a further embodiment of the present invention, the microbicidal agent may comprise a combination of bisabolol and Ciclopirox Olamine. These compositions are preferably encapsulated in the form of a foam, cream, wash, gel, suppository, ovule, ointment, film, tablet, foaming tablet, tampon, vaginal spray, or aerosol. The concentration of microbicidal agent will vary depending on the base or carrier. Preferably the concentration will fall within the parameters of approximately 0.01% to approximately 50% by weight, more preferably between approximately 0.01% to approximately 28%, and most preferably between approximately 0.05% and approximately 2%. In a preferred embodiment, the base or carrier is a gel and the microbicidal agent concentration will preferably range from approximately 0.01% to approximately 5% by weight, and more preferably from approximately 0.05% to approximately 2% by weight.

### BACKGROUND

1. Field of the Invention

The present invention relates generally to a device and method for the prevention or treatment of sexually transmitted infections and/or common vaginal infections, and more specifically to a microbicidal formulation containing a microbicidal agent.

2. Description of the Related Art

Sexually transmitted infections (STIs), referring to infections that are most often transmitted by direct sexual contact, remain an increasingly serious worldwide public health problem. These STIs, particularly viral infections, present a public health crisis. Women are especially at risk as many STIs are asymptomatic and there is a high morbidity rate associated with untreated infections.

Since its recognition in 1981, the acquired immunodeficiency syndrome (AIDS) has become a catastrophic pandemic. The AIDS pandemic is a premiere public health concern. Individuals who are at high risk of HIV infection are also at risk of infection by other sexually transmitted pathogens. Similarly, individuals at risk for non-HIV sexually transmitted pathogens are also at high risk for HIV infection.

Additionally, it is significant to note that women comprise the most rapidly increasing population of the AIDS epidemic. Sexual transmission of HIV in women occurs by infected semen being placed into the vagina, rectum, or other orifice. Currently, the only prevention strategy available for HIV/AIDS prevention is the condom.

Clinical pathologies attributable to STIs are profound. STIs cause acute and chronic infections, infertility, and in some cases cancer. Vaccines, which are costly and time-consuming to develop, are unavailable for HIV/AIDS prevention. HIV treatment employs therapeutic strategies, such as retrovirus triple therapy (e.g., AZT, DDI, etc.) to lower virus burden. However, this expense renders this therapeutic option practically unavailable to populations in developing countries where HIV is most prevalent. Indeed, the sum of all available STI therapeutics is effective against only a limited number of susceptible pathogens. Furthermore, this limited therapeutic arsenal is largely confined to proprietary formulations, which are costly for the afflicted to procure.

Common vaginal infections also pose an increasingly serious worldwide public health problem and can increase the risk of acquiring STIs. Vaginal candidiasis is the most common form of vaginitis, occurring more frequently than trichophyton, chlamydia, gonorrhea, or other bacterial infections. It is estimated that 75% of women will experience at least one episode of vulvovaginal candidiasis. Forty to 50% will experience a second episode in their life time. A much smaller (probably less than 5%), but still significant, number of women will suffer from repeated, often intractable attacks. Candidiasis is known to increase the risk of HIV acquisition. BV, previously known as nonspecific vaginitis or Gardnerella vaginitis, is the most common cause of vaginal discharge. It may be the cause of up to 50% of cases of vaginitis in all women and from 10-30% in pregnant women. BV is not a sexually transmitted disease although it is sometimes listed as one. However, the risk of contracting the disease increases with multiple sex partners.

Presently marketed vaginal contraceptive compositions, often containing nonoxynol-9 as an active ingredient, are generally known in the art. While presently marketed vaginal contraceptive formulations aid in preventing pregnancy, their ability to effectively prevent STIs, particularly HIV/AIDS, as well as oral rectal and vaginal infections is unclear. Nonoxynol-9 and other detergents as well as their compositions can destroy the natural and safe ecology of the vagina, such as by destroying lactobacillus bacteria. Further, spermicides may cause vaginal irritation, particularly with frequent exposure or higher doses.

Accordingly, there remains an urgent and compelling need for alternative methods and devices to prevent the transmission of or treat sexually transmitted infections, particularly HIV/AIDS, and/or common vaginal infections, while minimizing vaginal disruptions.

### SUMMARY OF THE INVENTION

To achieve the foregoing, and in accordance with the purposes of the present invention as embodied and broadly described herein, it is an object of this invention to provide microbicidal compounds and microbicidal agents that prevent the transmission of or treat sexually transmitted infections and/or common vaginal infections while minimizing disruptions to vaginal ecology.

In one aspect, the present invention includes vaginal microbicidal compositions suitable for preventing the transmission of sexually transmitted infections comprising the microbicidal agent bisabolol. Another embodiment of the present invention includes microbicidal compositions suitable for preventing the transmission of common vaginal infections comprising the microbicidal agent bisabolol. A further embodiment of the present invention includes microbicidal compositions suitable for treating sexually transmitted infections comprising the microbicidal agent bisabolol. In another embodiment of the present invention, the microbicidal compositions according to the present invention comprising the microbicidal agent bisabolol are suitable for treating common vaginal infections. These compositions are preferably encapsulated in the form of a foam, cream, wash, gel, suppository, ovule, ointment, film, tablet, foaming tablet, tampon, vaginal spray, or aerosol. In a further embodiment of the present invention, the microbicidal agent may comprise a combination of bisabolol and Ciclopirox Olamine.

The concentration of microbicidal agent will vary depending on the base or carrier. Preferably the concentration will fall within the parameters of approximately 0.01% to approximately 50% by weight, more preferably between approximately 0.01% to approximately 28%, and most preferably between approximately 0.05% and approximately 2%. In a preferred embodiment, the base or carrier is a gel and the microbicidal agent concentration will preferably range from approximately 0.01% to approximately 5% by weight, and more preferably from approximately 0.05% to approximately 2% by weight.

The present invention may further include methods of preventing conception and transmission of sexually transmitted infections by using microbicidal compositions according to the present invention by themselves or in conjunction with condoms, delivery devices, applicators, barrier-type devices and other vaginal or anorectal compositions.

### DETAILED DESCRIPTION OF THE PREFERRED EMBODIMENTS

A preferred embodiment of the present invention is now described. While specific configurations and arrangements are discussed, it should be understood that this is done for illustrative purposes only. A person skilled in the relevant art will recognize that other configurations and arrangements can be used without departing from the spirit and scope of the invention. It will be apparent to a person skilled in the relevant art that this invention can also be employed in a variety of other devices and applications.

According to the present invention, the protection from sexually transmitted infections, such as HIV/AIDS, and common vaginal infections, such as bacterial vaginosis and vaginal candidiasis, can be obtained by placing an antimicrobial composition or device in the vagina, rectum or other orifice, which can inactivate the virus, prevent or limit contact of the virus or its carrier cells with the epithelium or prevent or hinder its entry into the orifice.

According to the present invention, microbicidal compositions have been found to be useful for protection against and prevention of sexually transmitted infections. They may be used alone or in conjunction with delivery and/or contraceptive devices or methods, such as mechanical barrier-type devices (such as a diaphragm, cap, or sponge), vaginal contraceptives, intra-uterine-devices, rhythm, and a variety of applicators. Microbicidal compositions according to the present invention generally contain a microbicidal agent and a base or carrier, such as a foam, cream, wash, gel, suppository, ovule, ointment, film, tablet, foaming tablet, tampon, vaginal spray, or aerosol.

Microbicidal agents act according to a variety of mechanisms. Specifically, such agents may destroy microbes, prevent their pathogenic action, or inhibit their growth. In particular, non-cytotoxic microbicides act by preventing interactions of STI-causing viruses and bacteria with host cells, or otherwise prevent contact with host cells. Microbicidal agents, also referred to as anti-infective agents, may be applied topically to the skin and/or mucous membranes. Topical microbicidal agents may be directed at bacteria, viruses, fungi, and parasites. Such topical microbicidal agents are convenient for vaginal application and have been successfully employed in the prevention and treatment of a number of infections including some STIs in animal models. Preferable microbicidal agents inactivate bacteria and viruses, and are inexpensive, affordable, stable at ambient temperature, compatible and active after mixture with cosmetically acceptable formulations, non-toxic and non-damaging to vulvar, vaginal, cervical, penile or other epithelium.

The present invention provides microbicidal compounds and microbicidal agents that prevent the transmission of or treat sexually transmitted infections and/or common vaginal infections. Sexually transmitted infections include, but are not limited to, human immunodeficiency virus (HIV), herpes simplex virus type 1 (HSV-1), herpes simplex virus type 2 (HSV-2), gonorrhea, chlamydia, syphilis, and trichomoniasis. Common vaginal infections include, but are not limited to, bacterial vaginosis (BV) and vaginal candidiasis. Similar microbicidal compositions and methods of application of such compositions, as described herein, can be used for treating sexually transmitted infections and/or common vaginal infections and for preventing the transmission of sexually transmitted infections and/or common vaginal infections.

A preferred microbicidal agent according to the present invention is bisabolol. In particular, alpha-bisabolol (levomenol) is a preferred microbicidal agent. Bisabolol is a naturally occurring unsaturated monocyclic sesquiterpene alcohol. Alpha-bisabolol is used in a wide range or cosmetic formulations as a skin conditioning agent at concentrations ranging from approximately 0.001% in lipstick to approximately 1% in underarm deodorants. Bisabolol has been used for cosmetic purposes in India and worldwide in skin, oropharyngeal, nasal and vaginal products such as douches up to concentrations of approximately 5%, but has not been used in the prevention or treatment of STIs and/or common vaginal diseases. Bisabolol is the main active principle of chamomile (Matricaria chamomilla) and up to approximately 50% of the essential oil of chamomile consists of alpha-bisabolol. Bisabolol has shown little toxicity in oral and dermal toxicity, genotoxicity, reproductive/developmental toxicity, sensitization and photosensitization studies. Bisabolol has shown very high anti-HIV activity and other antimicrobial activity, and shows no effect on lactobacilli at concentrations of 2 mg/ml and 10 mg/ml

In preferred embodiments, the bisabolol of the present invention is 3-Cyclohexene-1-methanol, alpha,4-dimethyl-alpha-(4-methyl-3-pentenyl). Synonyms and other embodiments of the bisabolol of the present invention include alpha,4-Dimethyl-alpha-(4-methyl-3-pentenyl)-3-cyclohexene-1-methanol; 6-Methyl-2-(4-methyl-3-cyclohexen-1-yl)-5-hepten-2-ol; EINECS 208-205-9; EINECS 246-973-7; alpha, 4-Dimethyl-alpha-(4-methyl-3-pentenyl)-3-cyclohexene-1-methanol, and alpha-Bisabolol. Systematic names include (R*,R*)-(1)-alpha,4-Dimethyl-alpha-(4-methyl-3-pentenyl)cyclohex-3-ene-1-methanol; (R*,R*)-alpha,4-Dimethyl-alpha-(4-methyl-3-pentenyl)cyclohex-3-ene-1-methanol; 3-Cyclohexene-1-methanol, alpha,4-dimethyl-alpha-(4-methyl-3-pentenyl)-, (R*,R*)-(9CI); 3-Cyclohexene-1-methanol, alpha,4-dimethyl-alpha-(4-methyl-3-pentenyl)-, (alphaR, 1 R)-rel-3-Cyclohexene-1-methanol, alpha,4-dimethyl-alpha-(4-methyl-3-pentenyl)-, (theta,theta)-(+/−); 5-Hepten-2-ol, 6-methyl-2-(4-methyl-3-cyclohexen-1-yl).

Further synonyms and chemical derivates (typically isolated from plants; usually being isolated along with alpha bisabolol) of the bisabolol of the present invention include the following:
Bisabolol (−)
Bisabolol Alpha (+)
Bisabolol Alpha (−)
Bisabolol Alpha (DL)
Bisabolol Alpha (6R-7R)
Bisabolol Alpha 4 epi
Bisabolol Alpha 4 epi (+)
Bisabolo Alpha 6 (R)-7 (R): (+)
Bisabolol Alpha 6 (S)-7 (S): (−)
Bisabolol AR
Bisabolol AR: dihydro
Bisabolol Beta
Bisabolol Beta: 2 (R)—hydroxy: (1R, 7R)
Bisabolol Beta: 2 acetoxy
Bisabolol: epi
Bisabolol -1-on-13-oic acid methyl ester
Bisabolol oxide
Bisabolol oxide A
Bisabolol oxide A acetate, alpha
Bisabolol oxide B
Bisabolol oxide B alpha
Bisabolol oxide B, beta

In a preferred embodiment, the bisabolol of the present invention is a colorless clear viscous liquid having the following properties:
Formula: C15H260
CAS-Number: 515695
Formula weight: 22.4
Assay (GC area %): 97.4% REL

Bisabolol has been shown to inhibit HIV infectivity effectively when mixed with the virus for 5 minutes before washing (IC₅₀: 1 μg/ml) or when mixed with the virus and cells (IC₅₀: 0.6 μg/ml). In contrast to sulfonated polymers that are presently under development as microbicides, bisabolol inactivates HIV even when washed away after short-term treatment.

Bisabolol can be purchased in pure form isolated from natural sources in concentrations such as 95% to 97% or in an pure form produced synthetically in concentrations such as 85%. Other forms of bisabolol in differing concentrations could be utilized in the present invention, as would be apparent to one skilled in the relevant art. In a preferred embodiment, alpha-bisabolol can be synthesized by stirring ketodiene in ether, and then into a solution of methyl magnesium iodide at room temperature for two hours. Saturated aqueous ammonium acetate solution is then added, and the ether and aqueous layers are separated. Washing the aqueous phase with ether and evaporation of the combined ether washes extracts the alpha-bisabolol as colorless oil. Natural alpha-bisabolol containing a minimum of 95% active alpha-bisabolol isomer can be purchased from Pangaea Sciences, 2962 Saint Malo Circle, Mississauga, Ontario L5N1 S9. Dragosantol, an alpha-bisabolol, can be purchased from Dragoco, Inc., 317 West 13th Street, New York, N.Y. 10014.

In a further embodiment of the present invention, the microbicidal agent is a combination of bisabolol and Ciclopirox Olamine. Ciclopirox Olamine is a broad-spectrum hydroxypyridone antimycotic and antibacterial agent. It inhibits the growth of pathogenic dermatophytes, yeasts and Malassezia furfur. Ciclopirox Olamine exhibits fungicidal activity in vitro against isolates of Trichophyton rubrum, Trichophyton mentagrophytes, Epidermophyton floccosum, Microsporum canis and Candida albicans. It is prevents the apoptotic death of neuronal cells brought about by trauma or stroke. In a preferred embodiment, the Ciclopirox Olamine of the present invention is a white crystal powder having a molecular formula of C₁₂H₁₇NO₂ C₂H₇NO and a CAS Number of 41621-49-2. Synonyms include 6-Cyclohexyl-1-hydroxy-4-methyl-2-(1H)-pyridone ethanolamine salt.

Ciclopirox Olamine is available from sources such as Micro Labs Limited, P.O. Box No. 5061, 3 Queens Road, Bangalor 560 001. Ciclopirox olamine is used in various formulations including Loprox from Aventis Pharma Ltd., Aventis House, 50 Kings Hill Avenue, West Malling, Kent ME19 4AH; Dafnegin from CSC Pharmaceuticals; and is also available from Glenmark Pharmaceuticals Ltd., B/2, Mahalaxmi Chambers, 22, Bhulabhai Desai Road, Mumbai 400 026; Lyka Labs Ltd., 77 Nehru Road, Vile Parle (East), Mumbai, 400099, INDIA; and Zydus Cadila, Corporate Headquarters, Zydus Tower, Satellite Cross Roads, Ahmedabad 380015, INDIA. Ciclopirox olamine is further described in U.S. Pat. No. 3,883,545, which is incorporated herein by reference. In alternate embodiments, Ciclopirox Olamine may be supplied with a foam, cream, gel, suppository, lotion, ointment, film, tablet, or other base or carrier as would be apparent to one skilled in the relevant art.

Preferably, the present method involves the topical application of the microbicidal agent. In the context of the present invention, it is to be understood that the term topical application includes application to the body cavities as well as to the skin. Thus, in a preferred embodiment, the microbicidal agent is applied to a body cavity such as the vagina, anus, or mouth.

In a particularly preferred embodiment, the composition including the microbicidal agent, preferably bisabolol, is applied to the vagina. In a preferred embodiment, the topical application is carried out prior to the beginning of vaginal intercourse, preferably from 0 to 8 hours, more preferably from 0 to 60 minutes, and most preferably from 0 to 15 minutes.

The concentration of microbicidal agent will vary depending on the base or carrier. Preferably the concentration will fall within the parameters of approximately 0.01% to approximately 50% by weight, more preferably between approximately 0.01% to approximately 28%, and most preferably between approximately 0.05% and approximately 2%. Wherein the microbicidal composition comprises bisabolol and ciclopirox olamine, the concentration of the combined agent is preferably approximately 0.01% to approximately 50% by weight, more preferably between approximately 0.01% to approximately 28%, and most preferably between approximately 0.05% and approximately 2%. Preferably bisabolol and ciclopirox olamine are provided in equal concentrations within the combined agent, however each may comprise approximately 0.01% to approximately 99.9% of the total combined agent, for example the combined agent may comprise 60% bisabolol and 40% ciclopirox olamine.

The microbicidal agent may be applied to the vagina in a number of forms including but not limited to foam, cream, wash, gel, suppository, ovule, ointment, film, tablet, foaming tablet, tampon, vaginal spray, or aerosol. In a preferred embodiment, the base or carrier is a gel and the microbicidal agent concentration will preferably range from approximately 0.01% to approximately 5% by weight, and more preferably from approximately 0.05% to approximately 2% by weight.

The composition containing the bisabolol may be applied to the vagina in any conventional manner, as would be apparent to one skilled in the relevant art. Suitable devices for applying the composition to the vagina are disclosed in U.S. Pat. Nos. 3,826,828, 4,108,309, 4,360,013, and 4,589,880, which are incorporated herein by reference. In a preferred embodiment, the microbicidal composition may be applied to the vagina by an applicator. In a preferred embodiment, the applicator may be a tube from approximately 2.5 to approximately 25 centimeters in length, or more preferably approximately 5 to approximately 10 centimeters in length. In an alternate embodiment, the applicator may have one or more holes distributed regularly along its length. In alternate embodiments, the applicator may be a vaginal ring, or other slow release applicators, as would be apparent to one skilled in the relevant art.

In an alternate embodiment, the microbicidal composition may be provided in the form of a suppository, preferably a vaginal suppository.

In alternate embodiments, the microbicidal composition of the present invention may be topically administered to the rectum or anorectal area. The composition may be applied to the anus in a number of forms including but not limited to a foam, cream, jelly, or other application such as those described above with regard to vaginal application. In the case of anorectal application, it may be preferred to use an applicator, which distributes the composition substantially evenly throughout the anus. In a preferred embodiment, the applicator is a tube approximately 2.5 to approximately 25 cm in length, or more preferably approximately 5 to approximately 10 cm in length, and may include one or more holes distributed regularly along its length. In alternate embodiments, differing varieties of applicators could be used, or no applicator may be used, as would be apparent to one skilled in the relevant art.

In an alternate embodiment, a composition containing a microbicidal agent such as bisabolol may be delivered orally. Oral application is preferably carried out by providing the microbicidal composition in the form of a mouthwash or gargle. In one embodiment, oral application may be used to prevent infection during dental procedures. Suitably, the microbicidal composition is applied prior to the beginning of the dental procedure and periodically throughout the procedure. In the case of a mouthwash or gargle, it may be preferred to include in the composition an agent which will mask the taste and/or odor of the microbicidal agent such as bisabolol. Such agents include those flavoring agents typically found in mouthwashes and gargles, such as spearmint oil, cinnamon oil, or other flavoring agents.

In alternate embodiments, the microbicidal compound of the present invention may further include a vaginal contraceptive agent. Compositions including vaginal contraceptive agents are disclosed in U.S. Pat. Nos. 2,149,240, 2,330,846, 2,436,184, 2,467,884, 2,541,103, 2,623,839, 2,623,841, 3,062,715, 3,067,743, 3,108,043, 3,174,900, 3,244,589, 4,093,730, 4,187,286, 4,283,325, 4,321,277, 4,368,186, 4,371,518, 4,389,330, 4,415,585, and 4,551,148, which are incorporated herein by reference, and the present method may be carried out by applying bisabolol or other microbicidal agent to the vagina in the form of such a composition. Vaginal contraceptive agents are preferably not cytotoxic, such as cellulose sulfate or polystyrene sulfonate. However, cytotoxic contraceptive agents may also be used, such include nonylphenoxypolyoxyethylene glycol (nonoxynol 9), benzalkonium chloride, and chlorindanol. Suitably, the pH of the microbicidal composition is between approximately 3.5 to approximately 9, more preferably between approximately 3.5 to approximately 6, and most preferably approximately 4.

The present compositions may also be in the form of a time-release composition and slow-releasing devices. In such an embodiment, the microbicidal agent such as bisabolol is incorporated in a composition that will release bisabolol at a rate that will result in the vaginal or anorectal concentrations described above. Time-release and slow-release compositions are disclosed in U.S. Pat. Nos. 5,185,155; 5,248,700; 4,011,312; 3,887,699; 5,143,731; 3,640,741; 4,895,724; and 4,795,642, all of which are incorporated herein by reference.

The present compositions may be provided in a form that releases the microbicidal agent in response to an event such as vaginal or anorectal intercourse. For example, the microbicidal composition may contain bisabolol in vesicles or liposomes which are disrupted by the mechanical action of intercourse. Examples of compositions comprising liposomes are described in U.S. Pat. No. 5,231,112 which is incorporated herein by reference.

It should also be understood that the microbicidal compositions of the present invention may be associated with a device, such as an intrauterine device (IUD), vaginal dispenser vaginal ring, intravaginal barrier-type device, intravaginal sponge, or a condom. In the case of an IUD or diaphragm, time-release and/or mechanical-release compositions may be preferred, while in the case of condoms, mechanical-release compositions may be preferred. In alternate embodiments, the device may be an intravaginal sponge that comprises and releases bisabolol. Intravaginal sponges are disclosed in U.S. Pat. Nos. 3,916,898 and 4,360,013, which are incorporated herein by reference. The device may also be a vaginal dispenser that releases a bisabolol formulation. Vaginal dispensers are disclosed in U.S. Pat. No. 4,961,931, which is incorporated herein by reference. The device may be an intravaginal barrier-type device, such as those described in U.S. Pat. Nos. 4,858,624, 4,989,618, and 5,207,232, which are incorporated herein by reference. The device may also be a condom that is coated with bisabolol or a bisabolol formulation may be incorporated in the condom. In a preferred embodiment, the microbicidal agent comprises bisabolol, which is encapsulated in liposomes such that the bisabolol is released from the liposomes upon intercourse. The condom may be coated with other lubricants and penetration-enhancing agents such as those described in U.S. Pat. Nos. 4,537,776; 4,552,872; 4,557,934; 4,130,667, 3,989,816; 4,017,641; 4,954,487; 5,208,031; and 4,499,154, which are incorporated herein by reference. In an alternate embodiment, the microbicidal agent such as bisabolol may be contained inside the condom.

The size of the microbicidal composition will vary depending on the type of composition used. For example, when the composition is in the form of a suppository (including vaginal suppositories), the suppository will usually be approximately 1 to approximately 5 grams, preferably approximately 3 grams, and the entire suppository will be applied. A vaginal tablet will suitably be approximately 1 to approximately 5 grams, preferably approximately 2 grams, and the entire tablet will be applied. When the composition is vaginal cream, approximately 0.1 grams to approximately 10 grams may be applied, more preferably approximately 0.5 grams to approximately 5 grams, and most preferably approximately 3 grams to approximately five grams. When the composition is vaginal gel, approximately 0.1 grams to approximately 10 grams may be applied, more preferably approximately 0.5 grams to approximately 5 grams, and most preferably approximately 3 grams to approximately live grams. When the composition is a vaginal foam, approximately 0.1 to approximately 5 grams of the spray-foam may be applied, preferably approximately 0.5 grams to approximately three grams. When the composition is an anorectal cream, approximately 0.1 to approximately 5 grams may be applied, preferably approximately 0.5 grams to approximately 3 grams, most preferably approximately 2 grams to approximately 3 grams. When the composition is an anorectal foam, approximately 0.1 ml to approximately 10 ml of the spray-foam may be applied, more preferably approximately 3 ml to approximately 8 ml, and most preferably approximately 6 ml to approximately 7 ml. When the composition is a mouthwash or gargle, approximately 1 ml to approximately 20 ml may be applied, preferably approximately 8 ml to 10 ml, and most preferably approximately 10 ml.

While the invention has been particularly shown and described with reference to preferred embodiments thereof, it will be understood by those skilled in the art that various changes in form and details may be made therein without departing from the spirit and scope of the invention.

1. A microbicidal composition for preventing the transmission of a sexually transmitted infection comprising at least one microbicidal agent, wherein said at least one microbicidal agent comprises bisabolol.

2. The microbicidal composition of claim 1, wherein said at least one microbicidal agent comprises bisabolol and ciclopirox olamine.

3. The microbicidal composition of claim 1, further comprising a carrier selected from the group consisting of foam, cream, wash, gel, suppository, ovule, ointment, film, tablet, foaming tablet, tampon, vaginal spray and aerosol.

4. The microbicidal composition of claim 1, wherein said bisabolol is alpha-bisabolol (levomenol).

5. The microbicidal composition of claim 1, wherein said bisabolol is a bisabolol derivative.

6. The microbicidal composition of claim 1, wherein said bisabolol has the formula C₁₅H₂₆0.

7. The microbicidal composition of claim 1, wherein said bisabolol is isolated from natural sources.

8. The microbicidal composition of claim 1, wherein said bisabolol is produced synthetically.

9. The microbicidal composition of claim 1, further comprising chamomile.

10. The microbicidal composition of claim 1, comprising about 0.01% to about 50% by weight of said microbicidal agent.

11. The microbicidal composition of claim 10, comprising about 0.01% to about 28% by weight of said microbicidal agent.

12. The microbicidal composition of claim 11, comprising about 0.05% to about 2% by weight of said microbicidal agent.

13. The microbicidal composition of claim 1, further comprising a vaginal contraceptive agent.

14. The microbicidal composition of claim 13, wherein said vaginal contraceptive agent is non-cytotoxic.

15. The microbicidal composition of claim 14, wherein said vaginal contraceptive agent is cellulose sulfate.

16. The microbicidal composition of claim 14, wherein said vaginal contraceptive agent is polystyrene sulfonate.

17. The microbicidal composition of claim 13, wherein said vaginal contraceptive agent is cytotoxic.

18. The microbicidal composition of claim 17, wherein said vaginal contraceptive agent is selected from the group consisting of nonylphenoxypolyoxyethylene glycol (nonoxynol 9), benzalkonium chloride, and chlorindanol.

19. The microbicidal composition of claim 1, having a pH of between about 3.5 to about 9.

20. The microbicidal composition of claim 19, having a pH of between about 3.5 to about 6.

21. The microbicidal composition of claim 20, having a pH of about 4.

22. The microbicidal composition of claim 1, wherein said composition is a time-release composition.

23. The microbicidal composition of claim 1, wherein said sexually transmitted infection is selected from the group consisting of HIV, HSV-1, HSV-2, gonorrhea, chlamydia, syphilis, and trichomoniasis.

24. The microbicidal composition of claim 1, further comprising an applicator.

25. A device comprising the microbicidal composition of claim 1, wherein said device is selected from the group consisting of intrauterine device, vaginal dispenser, vaginal ring, intravaginal barrier-type device, intravaginal sponge, and condom.

26. A method of preventing the transmission of a sexually transmitted infection comprising topically applying a microbicidal composition comprising at least one microbicidal agent, wherein said microbicidal agent comprises bisabolol.

27. The method of claim 26, comprising applying said microbicidal composition to the skin.

28. The method of claim 26, comprising applying said microbicidal composition to a body cavity.

29. The method of claim 28, wherein said body cavity is the mouth.

30. The method of claim 28, wherein said body cavity is the anus.

31. The method of claim 28, wherein said body cavity is the vagina.

32. The method of claim 31, wherein said microbicidal composition is applied prior to the beginning of vaginal intercourse.

33. The method of claim 32, wherein said microbicidal composition is applied between about 0 and about 8 hours prior to the beginning of vaginal intercourse.

34. The method of claim 33, wherein said microbicidal composition is applied between about 0 and about 60 minutes prior to the beginning of vaginal intercourse.

35. The method of claim 34, wherein said microbicidal composition is applied between about 0 to about 15 minutes prior to the beginning of vaginal intercourse.

36. The method of claim 26, wherein said microbicidal composition is in a form selected from the group consisting of cream, wash, gel, suppository, ovule, ointment, film, tablet, foaming tablet, tampon, vaginal spray and aerosol.

37. The method of claim 26, comprising applying said microbicidal composition to the rectum.

38. The method of claim 26, comprising applying said microbicidal composition to the anorectal area.

39. The method of claim 26, further comprising using a contraceptive device.

40. The method of claim 39, wherein said contraceptive device is selected from the group consisting of intrauterine device, intravaginal barrier-type device, intravaginal sponge, and condom.

41. The method of claim 36, comprising applying about 1 to about 5 grams of said suppository.

42. The method of claim 41, comprising applying about 3 grams of said suppository.

43. The method of claim 36, comprising applying about 1 to about 5 grams of said tablet.

44. The method of claim 43, comprising applying about 2 grams of said tablet.

45. The method of claim 36, comprising applying about 0.1 to about 10 grams of said cream wherein said cream is a vaginal cream.

46. The method of claim 45, comprising applying about 0.5 to about 5 grams of said cream wherein said cream is a vaginal cream.

47. The method of claim 46, comprising applying about 3 to about 5 grams of said cream wherein said cream is a vaginal cream.

48. The method of claim 36, comprising applying about 0.1 to about 10 grams of said gel.

49. The method of claim 48, comprising applying about 0.5 to about 5 grams of said gel.

50. The method of claim 49, comprising applying about 3 to about 5 grams of said gel.

51. The method of claim 36, comprising applying about 0.1 to about 5 grams of said foam wherein said foam is a vaginal foam.

52. The method of claim 51, comprising applying about 0.5 to about 3 grams of said foam wherein said foam is a vaginal foam.

53. The method of claim 36, comprising applying about 0.1 to about 5 grams of said cream wherein said cream is an anorectal cream.

54. The method of claim 53, comprising applying about 0.5 to about 3 grams of said cream wherein said cream is an anorectal cream.

55. The method of claim 54, comprising applying about 2 to about 3 grams of said cream wherein said cream is an anorectal cream.

56. The method of claim 36, comprising applying about 0.1 to about 10 ml of said foam wherein said foam is an anorectal foam.

57. The method of claim 56, comprising applying about 3 to about 8 ml of said foam wherein said foam is an anorectal foam.

58. The method of claim 57, comprising applying about 6 to about 7 ml of said foam wherein said foam is an anorectal foam.

59. The method of claim 26, wherein said microbicidal agent is released upon sexual intercourse.

60. The method of claim 59, wherein said microbicidal agent is contained in vesicles.

61. The method of claim 59, wherein said microbicidal agent is contained in liposomes.

62. The method of claim 26, wherein said at least one microbicidal agent comprises bisabolol and ciclopirox olamine

63. The method of claim 26, wherein said microbicidal composition comprises about 0.01% to about 50% by weight of said microbicidal agent.

64. The method of claim 26, wherein said sexually transmitted infection is selected from the group consisting of HIV, HSV-1, HSV-2, gonorrhea, chlamydia, syphilis, and trichomoniasis.

65. A method of treating a sexually transmitted infection comprising topically applying a microbicidal composition comprising at least one microbicidal agent, wherein said microbicidal agent comprises bisabolol.

66. The method of claim 65, comprising applying said microbicidal composition to the skin.

67. The method of claim 65, comprising applying said microbicidal composition to a body cavity.

68. The method of claim 67, wherein said body cavity is the vagina.

69. The method of claim 65, wherein said microbicidal composition is in a form selected from the group consisting of foam, cream, wash, gel, suppository, ovule, ointment, film, tablet, foaming tablet, tampon, vaginal spray and aerosol.

70. The method of claim 65, wherein said at least one microbicidal agent comprises bisabolol and ciclopirox olamine

71. The method of claim 65, wherein said microbicidal composition comprises about 0.01% to about 50% by weight of said microbicidal agent.

72. The method of claim 65, wherein said sexually transmitted infection is selected from the group consisting of HIV, HSV-1, HSV-2, gonorrhea, chlamydia, syphilis, and trichomoniasis.

73. A microbicidal composition for preventing the transmission of a common vaginal infection comprising at least one microbicidal agent, wherein said at least one microbicidal agent comprises bisabolol.

74. The microbicidal composition of claim 73, wherein said at least one microbicidal agent comprises bisabolol and ciclopirox olamine.

75. The microbicidal composition of claim 73, further comprising a carrier selected from the group consisting of foam, cream, wash, gel, suppository, ovule, ointment, film, tablet, foaming tablet, tampon, vaginal spray and aerosol.

76. The microbicidal composition of claim 73, comprising about 0.01% to about 50% by weight of said microbicidal agent.

77. The microbicidal composition of claim 73, wherein said common vaginal infection is bacterial vaginosis.

78. The microbicidal composition of claim 73, wherein said common vaginal infection is vaginal candidiasis.

79. A method of preventing the transmission of a common vaginal infection comprising topically applying a microbicidal composition comprising at least one microbicidal agent, wherein said microbicidal agent comprises bisabolol.

80. The method of claim 79, comprising applying said microbicidal composition to the skin.

81. The method of claim 79, comprising applying said microbicidal composition to the vagina.

82. The method of claim 79, wherein said microbicidal composition is in a form selected from the group consisting of foam, cream, wash, gel, suppository, ovule, ointment, film, tablet, foaming tablet, tampon, vaginal spray and aerosol.

83. The method of claim 79, wherein said at least one microbicidal agent comprises bisabolol and ciclopirox olamine

84. The method of claim 79, wherein said microbicidal composition comprises about 0.01% to about 50% by weight of said microbicidal agent.

85. The method of claim 79, wherein said common vaginal infection is bacterial vaginosis.

86. The method of claim 79, wherein said common vaginal infection is vaginal candidiasis.

87. A method of treating common vaginal infections comprising topically applying a microbicidal composition comprising at least one microbicidal agent, wherein said microbicidal agent comprises bisabolol.

88. The method of claim 87, comprising applying said microbicidal composition to the skin.

89. The method of claim 87, comprising applying said microbicidal composition to the vagina.

90. The method of claim 87, wherein said microbicidal composition is in a form selected from the group consisting of foam, cream, wash, gel, suppository, ovule, ointment, film, tablet, foaming tablet, tampon, vaginal spray and aerosol.

91. The method of claim 87, wherein said at least one microbicidal agent comprises bisabolol and ciclopirox olamine

92. The method of claim 87, wherein said microbicidal composition comprises about 0.01% to about 50% by weight of said microbicidal agent.

93. The method of claim 87, wherein said common vaginal infection is bacterial vaginosis.

94. The method of claim 87, wherein said common vaginal infection is vaginal candidiasis.
